(19) United States
(12) Patent Application Publication
(10) Pub. No.: US 2026/0162554 A1
(43) Pub. Date: Jun. 11, 2026

(54) METHOD AND APPARATUS FOR COMPUTING PROJECTION INDEX IN RORSCHACH TEST BY COMBINING BIOMETRIC INDICATORS BASED ON ARTIFICIAL INTELLIGENCE

(71) Applicant: KOREA UNIVERSITY RESEARCH AND BUSINESS FOUNDATION, Seoul (KR)

(72) Inventors: Young-gun KO, Seoul (KR); Jin-young KIM, Seoul (KR); Haewon MIN, Seoul (KR); Won Mo KANG, Seoul (KR); Youjin PARK, Seoul (KR)

(21) Appl. No.: 19/409,525

(22) Filed: Dec. 4, 2025

(30) Foreign Application Priority Data
Dec. 5, 2024 (KR) 10-2024-0179226
Mar. 6, 2025 (KR) 10-2025-0029126

(51) Int. Cl. G09B 7/02; A61B 5/0205; G06F 3/01; G06F 40/284; G06V 40/20; G10L 15/26

(52) U.S. Cl. CPC G09B 7/02; A61B 5/02055; G06F 3/013; G06F 40/284; G06V 40/28; G10L 15/26

(57) ABSTRACT

A Rorschach test projection index computing apparatus according to an embodiment may perform an operation of acquiring a response of an examinee including a verbal response, a behavioral response, and a biometric indicator, while the examinee performs a Rorschach test; an operation of generating indicator data obtained by quantitatively converting each of the verbal response, behavioral response, and biometric indicator of the examinee; an operation of determining a statistical distribution position of the response of the examinee by comparing the indicator data with previously constructed normative data where a plurality of responses to the Rorschach test is configured as statistical data; and an operation of generating a projection index that indicates whether the response of the examinee is due to physical characteristics of a card or psychological projection as a numerical value from the indicator data and the statistical distribution position using a previously learned neural network model.

### CROSS-REFERENCE TO RELATED APPLICATION

This application claims priority under 35 U.S.C § 119 to Korean Patent Application No. 10-2024-0179226 filed on Dec. 5, 2024 in the Korean Intellectual Property Office, and Korean Patent Application No. 10-2025-0029126 filed on Mar. 6, 2025 in the Korean Intellectual Property Office the entire contents of which are hereby incorporated by reference.

### BACKGROUND OF THE INVENTION

### Field of the Invention

The present invention relates to a psychological analysis technique based on artificial intelligence, and more specifically, to a technique of computing a projection index by collecting verbal and behavioral responses and biometric indicators of an examinee when performing a Rorschach test and analyzing them on the basis of artificial intelligence.

Background of the Related Art The Rorschach test is a representative method of projective psychological test, and as the test may infer the internal psychological state from the responses of an examinee, it is useful in a situation where in-depth psychoanalysis is required. This test developed by Swiss psychiatrist Hermann Rorschach is conducted in a way of analyzing free responses of an examinee to a stimulus card using ink blots, and it is still widely used in the field of clinical psychology and mental health.

In the conventional Rorschach test method, a clinical expert is in charge of conducting the test, and a process of recording and analyzing responses by the examinee himself/herself is performed. When the examinee describes what he or she sees on the presented card, the examiner evaluates the responses according to specific scoring criteria and proceeds interpretation in a way of inferring personality traits of the examinee. This procedure uses verbal responses of the examinee as a major analysis element, and physiological responses or nonverbal expressions other than the verbal responses depend on the subjective observation of the examiner in many cases. Although some computerized scoring systems have been developed recently to enhance the objectivity of test results, they still remain at a level that does not significantly deviate from the conventional method.

Accordingly, there are some limitations in the conventional Rorschach test method. First, consistency of the results is likely to vary according to the skill level of the examiner. Although the Rorschach test scores responses of an examinee according to specific criteria, even identical responses may be interpreted in a different way according to the experience and determination method of the examiner.

In addition, the analysis method relying only on verbal reports may not fully reflect the psychological state of the examinee. When the examinee does not accurately express his or her emotion or intentionally avoids specific responses, reliability of test results may be lowered.

In addition, as the physical responses or physiological indicators of the examinee are not utilized in the test process, there is a problem in that nonverbal cues that the examinee shows are not properly reflected in the psychological interpretation process.

To overcome these limitations, it needs to increase the reliability and accuracy of projective responses by measuring and comprehensively analyzing biometric indicators of an examinee, together with verbal and behavioral responses of the examinee. Through these technical improvements, the Rorschach test may be developed as a more objective and quantitative evaluation method than before and may become as a more reliable psychological assessment tool.

### PRIOR ART

### Patent Document

Korean Patent Registration No. 10-1046269

### Non-Patent Document

Wongvorachan, T. (2023). Review of Rorschach Ink-Blot Test: Performance Assessment System (R-PAS®). Preprint, https://doi.org/10.31234/osf.io/jts4z

### SUMMARY OF THE INVENTION

Therefore, the present invention has been made in view of the above problems, and it is an object of the present invention to provide a technique of comprehensively analyzing verbal and behavioral responses and biometric indicators so that projective interpretation of the Rorschach test can be performed in a more objective and quantitative way.

Specifically, an object of the present invention is to implement a technique of analyzing both the biometric indicators and behavioral responses collected in real time and converting them into quantitative projection indexes by utilizing a machine learning model, together with conventional methods of recording verbal responses.

Through this, the major task of the present invention is to provide a more reliable psychological assessment method by reducing dependence on the subjective interpretation of an examiner and automating the implementation, scoring, and interpretation of the Rorschach test, for utilization of the Rorschach test based on projection indexes.

Meanwhile, the technical problems of the present invention are not limited to the technical problems mentioned above, and unmentioned other technical problems can be clearly understood by those skilled in the art from the following description.

To accomplish the above objects, according to one aspect of the present invention, there is provided a method performed by a Rorschach test projection index computing apparatus operated by a processor, the method comprising: an operation of acquiring a response of an examinee including a verbal response, a behavioral response, and a biometric indicator, while the examinee performs a Rorschach test; an operation of generating indicator data obtained by quantitatively converting each of the verbal response, behavioral response, and biometric indicator of the examinee; an operation of determining a statistical distribution position of the response of the examinee by comparing the indicator data with previously constructed normative data where a plurality of responses to the Rorschach test is configured as statistical data; and an operation of generating a projection index that indicates whether the response of the examinee is due to physical characteristics of a card or psychological projection as a numerical value from the indicator data and the statistical distribution position using a previously learned neural network model.

In addition, the operation of acquiring a response of an examinee may include an operation of presenting a Rorschach card to the examinee and acquiring an answer to what the presented card looks like, and additionally presenting preset questions about the presented card and acquiring responses to the questions.

In addition, the operation of acquiring a response of an examinee may include: an operation of converting voices of the examinee into text data through voice recognition and natural language processing as the verbal response; an operation of measuring a gaze pattern of the examinee, whether the card is rotated, and a response time using a webcam to acquire the behavioral response; and an operation of measuring biometric indicators including electromyography, skin conductance response, peripheral body temperature, heart rate, respiration rate, electroencephalography, and eye movement of the examinee using biometric sensors to acquire the biometric indicators.

In addition, the operation of generating indicator data may include: an operation of generating the indicator data by tokenizing the text data into units of morphemes using a morphological analyzer, tagging each token with a part of speech, and removing particles and conjunctions; an operation of detecting behavioral patterns including eyebrow movement, lip movement, eye blink, eye movement, pupil dilation, facial movement, neck movement, upper body movement, and lower body movement set in advance among the behavioral response using an image analysis technique; and an operation of generating indicator data by converting the biometric indicators into quantitative values using a signal processing technique.

In addition, the operation of evaluating a statistical distribution position may include: an operation of collecting response data including a verbal response, a behavioral response, and a biometric indicator from a preset number of experimenters or more for each card included in the Rorschach test; and an operation of previously constructing normative data by converting a probability of specific response data to be appeared on each Rorschach card into a percentile or a standard score on the basis of the collected response data.

In addition, the operation of evaluating a statistical distribution position may include: an operation of comparing a similarity between the response of the examinee and those of a specific group using data on a normal group and a group having specific psychological characteristics included in the normative data; an operation of comparing the indicator data of the examinee by converting it into a normal distribution or a percentile score of the normative data; and an operation of outputting how much the response of the examinee is different from an average response pattern of the normal group on the basis of a result of the comparison.

In addition, the operation of generating a projection index may include: an operation of receiving the indicator data and the statistical distribution position and comparing them with the normative data, by the neural network model; an operation of computing a probability value of whether the response of the examinee is due to the physical characteristics of the card or the psychological projection on the basis of the normative data; and an operation of computing a projection index of the examinee by converting the probability value into a percentile or a standard score.

In addition, the operation of computing a probability value may include: an operation of converting the indicator data of the examinee into a feature vector; an operation of computing a probability of a response that may appear on a specific card by comparing the feature vector with existing indicator data of the examinee included in the normative data; and an operation of evaluating whether the response of the examinee is a general card characteristic response or a psychological projective response on the basis of a result of the comparison.

In addition, the operation of computing a projection index may include: an operation of analyzing a statistical position of the projection index of the examinee with reference to the percentile information or standard score of the statistical projection index of a specific Rorschach card from the normative data; an operation of computing a final projection index by comprehensively evaluating a test result of each card and all test results on the basis of the analyzed data; and an operation of providing the computed projection index as a numerical result so that the examiner may interpret.

According to another embodiment, there is provided a Rorschach test projection index computing apparatus comprising: a memory including instructions; and a processor that performs a predetermined operation based on the instructions, wherein the operation of the processor includes: an operation of acquiring a response of an examinee including a verbal response, a behavioral response, and a biometric indicator, while the examinee performs a Rorschach test; an operation of generating indicator data obtained by quantitatively converting each of the verbal response, behavioral response, and biometric indicator of the examinee; an operation of determining a statistical distribution position of the response of the examinee by comparing the indicator data with previously constructed normative data where a plurality of responses to the Rorschach test is configured as statistical data; and an operation of generating a projection index that indicates whether the response of the examinee is due to physical characteristics of a card or psychological projection as a numerical value from the indicator data and the statistical distribution position using a previously learned neural network model.

The present invention may make a more objective and quantitative psychological assessment by automating the implementation, scoring, and interpretation of the Rorschach test to utilize the Rorschach test based on projection index. While conventional test methods greatly rely on subjective interpretation of an examiner, the present invention may quantitatively compute projection indexes by comprehensively analyzing biometric indicators and verbal and behavioral responses of an examinee.

In addition, as the projection indexes computed through the present invention can be used for more precise analysis of psychological characteristics compared with conventional subjective assessment methods, reliability and consistency of the test results can be improved, and based on this, more scientific diagnosis and intervention can be made without intervention of an expert in the field of mental health management and psychotherapy.

Through this, the present invention promotes popularization of the psychological assessment and allows many people to receive more reliable psychological analysis.

Meanwhile, the effects of the present invention are not limited to those mentioned above, and unmentioned other technical effects will be clearly understood by those skilled in the art from the following descriptions.

### BRIEF DESCRIPTION OF THE DRAWINGS

FIG. 1 is a view showing the configuration of a Rorschach test projection index computing apparatus according to an embodiment.

FIG. 2 is a flowchart illustrating the steps of the operation performed by a Rorschach test projection index computing apparatus according to an embodiment.

FIG. 3 is an exemplarily view showing an operation of acquiring verbal responses of an examinee using a microphone according to an embodiment.

FIG. 4 is an exemplarily view showing an operation of acquiring behavioral responses of an examinee using a webcam according to an embodiment.

FIG. 5 is an exemplary view showing an operation of acquiring biometric indicators of an examinee using various biosensors according to an embodiment.

### DETAILED DESCRIPTION OF THE PREFERRED EMBODIMENT

Details of the objects and technical configurations of the present invention and operational effects according thereto will be more clearly understood by the following detailed description based on the drawings attached in the specification of the present invention. An embodiment according to the present invention will be described in detail with reference to the accompanying drawings.

The embodiments disclosed in this specification should not be construed or used as limiting the scope of the present invention. For those skilled in the art, it is natural that the description including the embodiments of the present specification have various applications. Accordingly, any embodiments described in the detailed description of the present invention are illustrative for better describing of the present invention, and are not intended to limit the scope of the present invention to the embodiments.

The functional blocks shown in the drawings and described below are merely examples of possible implementations. Other functional blocks may be used in other implementations without departing from the spirit and scope of the detailed description. In addition, although one or more functional blocks of the present invention are expressed as separate blocks, one or more of the functional blocks of the present invention may be combinations of various hardware and software configurations that perform the same function.

In addition, the expressions including certain components are expressions of “open type” and only refer to existence of corresponding components, and should not be construed as excluding additional components.

Furthermore, when a certain component is referred to as being “connected” or “coupled” to another component, it may be directly connected or coupled to another component, but it should be understood that other components may exist in between.

Hereinafter, various embodiments of the present invention will be described with reference to the accompanying drawings. However, it should be understood that this is not intended to limit the present invention to specific embodiments, but to include various modifications, equivalents, and/or alternatives of the embodiments of the present invention.

The present invention proposes a Rorschach test projection index computing apparatus 100 for implementing a technique of collecting biometric indicators and verbal and behavioral response data of an examinee when performing a Rorschach test, analyzing the data on the basis of artificial intelligence, and computing projection indexes.

Hereinafter, the configuration of the Rorschach test projection index computing apparatus 100 of the present invention and the operation of each component will be described.

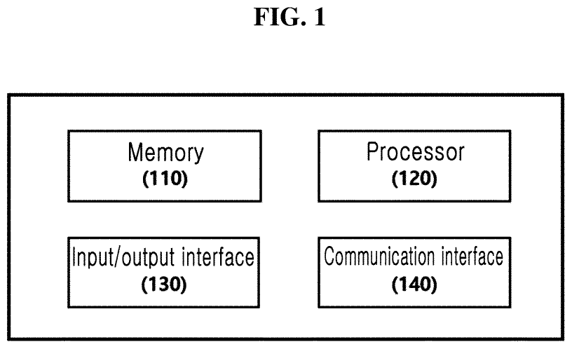

FIG. 1 is a view showing the configuration of the Rorschach test projection index computing apparatus 100 according to an embodiment (hereinafter, referred to as an ‘apparatus 100’).

Referring to FIG. 1, the apparatus 100 according to an embodiment may include a memory 110, a processor 120, an input/output interface 130, and a communication interface 140.

The memory 110 may store data acquired from an external device or data generated by itself. The memory 110 may store instructions that may perform the operation of the processor 120. For example, the memory 110 may store various types of information related to an examinee, a neural network model, and the like described below.

The processor 120 is a computing device that controls the overall operation. The processor 120 may execute instructions stored in the memory 110. The operation of the apparatus 100 according to the embodiment of this document may be understood as an operation performed by the processor 120.

The input/output interface 130 may include a hardware interface or a software interface for inputting or outputting information.

The communication interface 140 allows to transmit and receive information through a communication network. To this end, the communication interface 140 may include a wireless communication module or a wired communication module.

In addition, the apparatus 100 may be connected to a voice recognition device (e.g., microphone), an image recognition device (e.g., webcam), and a biometric indicator detection device (e.g., EEG sensor, electrode cap, thermometer, heart rate sensor, eye tracking device, etc.) to acquire response data including verbal responses, behavioral responses, and biometric indicators of the examinee described below.

The apparatus 100 may be implemented in various types of apparatuses that can perform an operation through the processor 120 and transmit and receive information through a network. For example, although the apparatus may be implemented as a server, a computer apparatus, a portable communication apparatus, a smart phone, a portable multimedia apparatus, a laptop computer, a tablet PC, or the like, it is not limited to these examples.

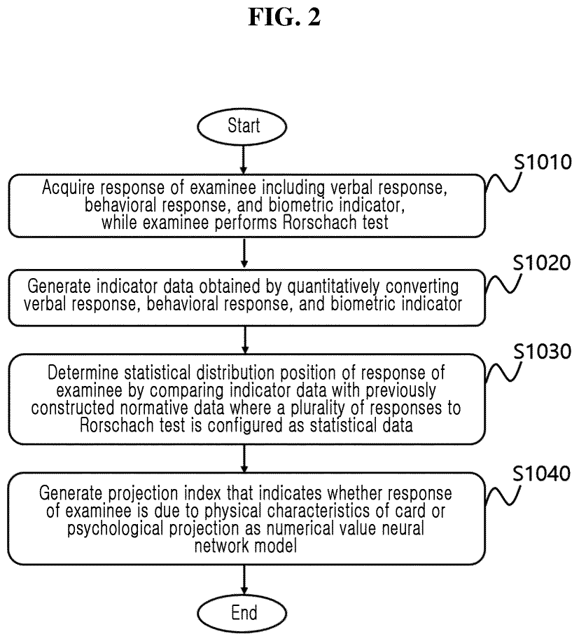

The apparatus 100 performs a Rorschach test on an examinee on the basis of the operation of FIG. 2 described below. At this point, the examinee may visit the location where the apparatus 100 is located by himself or herself and perform the Rorschach test offline according to the operation of the apparatus 100 shown in FIG. 2.

In addition, the examinee may access the apparatus 100 online and conduct the Rorschach test online. For example, the examinee may access the apparatus 100 online using his/her PC, laptop, tablet PC, smartphone, or the like, and conduct the Rorschach test after authentication (e.g., logging in) according to the operation of the apparatus 100 shown in FIG. 2.

At this point, the operation of the apparatus 100 is automated as shown in FIG. 2 described below so that the Rorschach test may be performed both offline and online through an image UI, natural language processing, and machine learning without an examiner. Meanwhile, in the ‘face-to-face’ Rorschach test method for projective interpretation, the examiner may supplement information collected by an automatic execution system through supplementary questions.

FIG. 2 is a flowchart illustrating the operation performed by the apparatus 100 according to an embodiment. The operation of the apparatus 100 according to the embodiment of FIG. 2 may be understood as an operation performed by the processor 120.

Each step disclosed in FIG. 2 is merely a preferred embodiment in achieving the objects of the present invention, and some steps may be added or deleted as needed, and any one step may be included and performed in another step. The order of each operation disclosed in FIG. 2 is arranged only for convenience of understanding, and this order is not limited to a time-series order, and the order may be changed to operate in a different way according to the choice of the designer.

Referring to FIG. 2, at step S1010, the apparatus 100 may acquire responses of an examinee including verbal responses, behavioral responses, and biometric indicators, while the examinee performs a Rorschach test.

Here, the Rorschach test is a representative method of projective psychological test, and is conducted in a way of analyzing free responses of the examinee to a plurality of cards using ink blots.

For example, the apparatus 100 may present a Rorschach card to the examinee and acquire an answer to what the examinee has seen on the card, and additionally present preset questions about the presented card and acquire information on the interpretation process and the way of responding of the examinee.

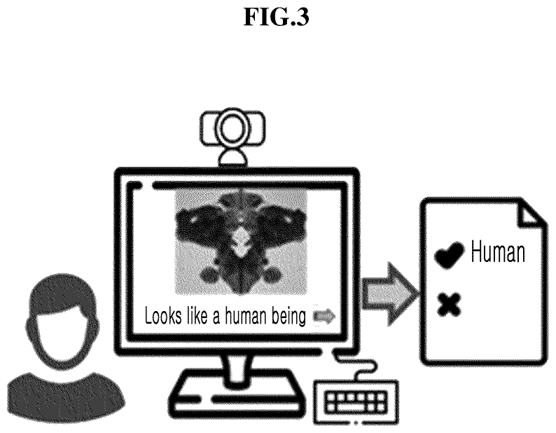

Meanwhile, unlike the conventional Rorschach test, the present invention simultaneously acquires and utilizes verbal responses, behavioral responses, and biometric indicators so that projective interpretation of the Rorschach test can be performed in a more objective and quantitative way. Examples of acquiring the verbal responses, behavioral responses, and biometric indicators during the Rorschach test are described below with reference to FIGS. 3 to 5.

FIG. 3 is an exemplarily view showing an operation of acquiring verbal responses of an examinee using a microphone according to an embodiment.

Referring to FIG. 3, in the process of acquiring verbal responses of an examinee, the apparatus 100 may acquire verbal responses of the examinee using a microphone and convert the speech of the examinee into text data using a voice recognition technique. At this point, the apparatus 100 may store phonetic features such as the speed of speech, changes in intonation, frequency of hesitation, and the like, in addition to conversion of voice into text. The apparatus 100 may evaluate whether a specific psychological tendency is revealed in the response of the examinee by performing morphological analysis, sentiment analysis, and keyword extraction by applying a natural language processing technique to the converted text. For example, when words containing negative sentiments such as “dark” and “scary” appear frequently in the response, it is possible to evaluate that the response is related to psychological anxiety. In addition, the apparatus 100 may quantitatively evaluate whether the response is only a simple description or includes a more complex thinking process by analyzing the structural complexity of a sentence.

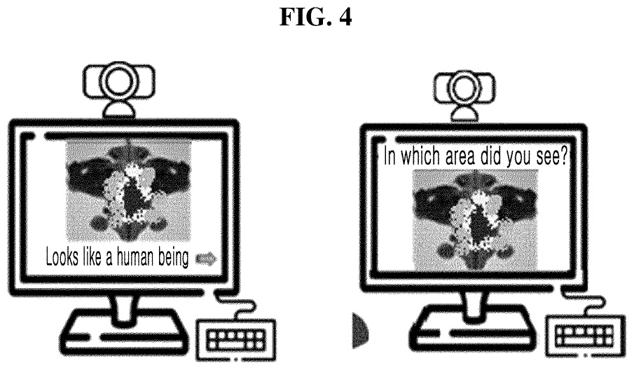

FIG. 4 is an exemplarily view showing an operation of acquiring behavioral responses of an examinee using a webcam according to an embodiment.

Referring to FIG. 4, in the process of acquiring a behavioral response of an examinee, the apparatus 100 may analyze eye tracking and gaze pattern using a webcam. Through this, intensity of the response to a specific stimulus element can be analyzed by evaluating on which area of a particular card the examinee focuses and whether there is an area where the gaze time is long. In addition, the apparatus 100 may evaluate whether the examinee moves the card and attempts a new interpretation by detecting whether the card is rotated and the rotation angle, and analyze whether there is a difference in the speed of response to a particular card of the examinee compared to those of other cards by measuring the response time taken to the response.

In addition, the apparatus 100 may collect biometric information including facial expressions, eye tracking, body movements, pulse, voice, language, and the like of the examinee using a webcam, and utilize the collected biometric information in an integrated manner by analyzing the information through a large language model (LLM) or a large multimodal model (LMM).

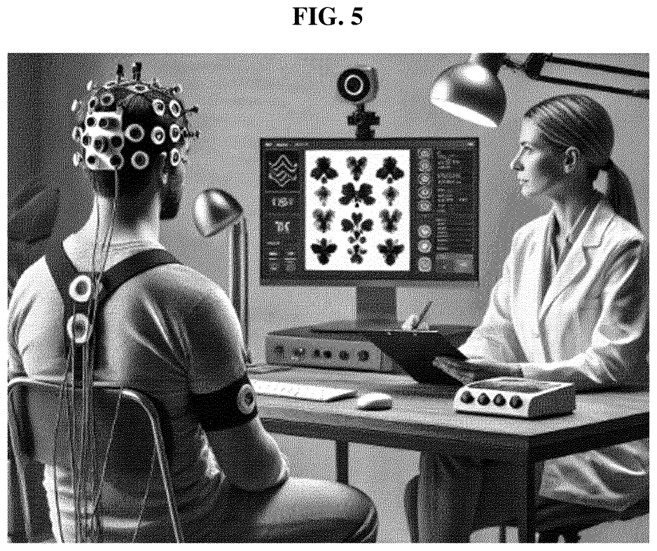

FIG. 5 is an exemplary view showing an operation of acquiring biometric indicators of an examinee using various biosensors according to an embodiment.

Referring to FIG. 5, in the process of acquiring biometric indicators of an examinees, the apparatus 100 may quantitatively analyze the emotional and physiological responses of the examinee by measuring various physiological responses using biometric sensors. To this end, the apparatus 100 may measure biometric indicators including electromyography (EMG), skin conductance response, peripheral body temperature, heart rate, respiration rate, electroencephalography (EEG), and eye movement in real time, and quantitatively analyze the biometric indicators.

For example, the apparatus 100 may attach electrodes to the frontalis muscle and the proximal part of the brachioradialis muscle of the forearm of the examinee to measure electromyographic responses, convert muscle activities into electrical signals, and record and utilize the electrical signals to evaluate how strongly the examinee emotionally responses to a particular card.

In addition, the apparatus 100 may attach electrodermal sensors to the index and ring fingers of the examinee to measure skin conductance responses, detect minute changes in the skin conductance, and utilize the changes to analyze the tension response to a particular card.

In addition, the apparatus 100 may attach a thermometer to the little finger of the examinee to measure peripheral body temperature, and analyze sympathetic nerve activities through changes in the peripheral body temperature.

In addition, the apparatus 100 may attach a heart rate sensor to the middle finger of the examinee to measure the heart rate and evaluate intensity of the psychological burden by tracking changes in the heart rate.

In addition, the apparatus 100 may evaluate psychological tension and relaxation state by analyzing changes in the breathing pattern of the examinee from piezo belts worn on the examinee to measure the respiratory rate.

In addition, to measure the electroencephalography (EEG), the apparatus 100 may analyze brain waves of the examinee by converting analog signals received from the scalp using an electrode cap worn on the examinee into digital signals. The apparatus 100 may utilize a total of 32 electrodes including two ground electrodes (Fpz, Oz), in addition to 30 major measurement areas (Fp1, Fp2, F7, F8, F3, F4, Fz, T3, T4, C3, C4, Cz, T5, T6, P3, P4, Pz, O1, O2, FTC1, FTC2, TCP1, TCP2, TT1, TT2, CP1, CP2, PO1, PO2, Oz), on the basis of the international 10-20 system. At this point, reference electrodes are attached to both earlobes (A1, A2), and changes in brain potential may be quantitatively analyzed in units of microvolts. In the brain wave analysis process, Power Spectrum is computed after the signal is converted into the frequency domain by applying Fast Fourier Transform (FFT), and this is divided into frequency bands of Delta (0-3 Hz), Theta (4-7 Hz), Alpha (8-13 Hz), and Beta (14-30 Hz), and input into the Brain Map (Delta Map, Theta Map, Alpha Map, Beta Map) to be analyzed. Accordingly, the measured brain waves may be utilized to evaluate how cognitively burdensome or meaningful the stimulus of a particular card is.

In addition, the apparatus 100 may analyze eye movement, including gaze shift and gaze pattern of the examinee, by utilizing an eye tracker. The eye tracker may detect an area on a particular card where the gaze of the examinee is focused, and measure the gaze time. Information on the eye movement measured through this process makes it possible to generate quantitative data and make in-depth interpretation of whether the response of the examinee is due to the physical characteristics of a particular card or a psychological projection.

At step S1020, the apparatus 100 may generate indicator data by quantitatively converting verbal responses, behavioral responses, and biometric indicators of the examinee. Through this, the apparatus 100 converts various response data measured from the examinee into numerical indicator data so that the neural network model described below may analyze the indicator data.

For example, to quantify the verbal responses, the apparatus 100 may convert speech of the examinee into text data using a morphological analyzer, tokenize the text data into units of morphemes, and tag each token with a part of speech. In this process, the apparatus 100 may generate indicator data to enhance accuracy of analysis by removing particles and conjunctions, and perform analysis of sentiments and semantic patterns by extracting major keywords. Through this, the neural network model described below may evaluate whether a specific emotional element or thinking pattern is included in the response of the examinee.

For example, to quantify the behavioral responses, the apparatus 100 may detect specific behavioral patterns of the examinee and convert them into indicator data by utilizing an image analysis technique. For example, eyebrow movement, lip movement, eye blink, eye movement, pupil dilation, facial movement, neck movement, upper body movement, lower body movement, and the like may be analyzed. Based on this, the neural network model described below may evaluate the emotional state or psychological tension of the examinee from nonverbal expressions.

For example, to quantify the biometric indicators, the apparatus 100 may convert biometric indicators into quantitative values by applying a signal processing technique. For example, data such as fluctuation patterns of heart rate, changes in the skin conductance, changes in the voltage of electromyography signals, fluctuation of respiratory rate, and changes in the pupil size may be measured in real time, and the data may be converted into quantitative indicators so that the neural network model described below may analyze the physiological responses of the examinee.

Meanwhile, examples of the quantified verbal responses, behavioral responses, and biometric indicators are shown below in Tables 1 to 3.

TABLE 1

| | | | Measured | |
|---|---|---|---|---|
| Response | Measurement | | psychological | Examples of |
| classification | items | Definitions | factors | situations |
| Biomedical | Fixation | Duration of fixing | Attention, | Pay attention to a |
| information | duration | eyes on a specific | Concentration | specific area |
| Eye | | location target | | |
| tracking | | | | |
| Biomedical | Number | Number of times | Attention, | See a specific area |
| information | of fixations | of shifting eyes to a | Interest | several times |
| Eye | | specific target | | |
| tracking | | | | |
| Biomedical | Time to | Time of fixing | Attention | Time of gazing at |
| information | first | eyes at a specific | | a specific target first |
| Eye | fixation | target first | | |
| tracking | | | | |
| Biomedical | Entry | Time required for | Attention | Time required for |
| information | time | eyes to enter a specific | | eyes to arrive at a |
| Eye | | area | | specific area |
| tracking | | | | |
| Biomedical | Fixation | Ratio of duration | Concentration, | Analyze ratio of |
| information | time ratio | of fixing eyes to total | Distraction | gazing at a specific |
| Eye | | time | | area out of the entire |
| tracking | | | | area |
| Biomedical | End time | Time of leaving | Attention | Analyze time of |
| information | | eyes from a specific | | leaving eyes from a |
| Eye | | target | | specific area |
| tracking | | | | |
| Biomedical | Dwell | Duration of | Strong | Case of fixing |
| information | time | dwelling eyes on a | interest | eyes at a specific area |
| Eye | | specific target | | for a long time |
| tracking | | | | |
| Biomedical | Dwell | Ratio of fixing | Attention, | Compute ratio of |
| information | time ratio | eyes on a specific | Concentration | fixing eyes at a |
| Eye | | target to total time of | | specific area |
| tracking | | seeing | | |
| Biomedical | Saccade | Number of times | Search, | Shift eyes from |
| information | count | of shifting eyes to | Anxiety, | one area to another |
| Eye | | another target | Tension, | area while searching, |
| tracking | | | Avoidance | Shift eyes for |
| | | | | avoidance |
| Biomedical | Saccadic | Average speed of | Efficiency | Rapidly shift eyes |
| information | velocity | shifting eyes | of information | during information |
| Eye | | | search | search |
| tracking | | | | |
| Biomedical | Saccadic | Average distance | Concentration | Distance of |
| information | amplitude | between eye shifts | of eye shift | shifting eyes |
| Eye | | | | comparing two targets |
| tracking | | | | spaced apart |
| Biomedical | Diameter | Change in the size | Tension, | Dilation of pupils |
| information | of pupil | of pupils | Interest | when seeing an |
| Eye | | | | interesting target |
| tracking | | | | |
| Biomedical | Eye shift | Direction and | Search, | Rapidly gazing at |
| information | | pattern of eyes | Confusion | a specific point |
| Eye | | | | on the map |
| tracking | | | | |

TABLE 2

| | | | | When one does |
|---|---|---|---|---|
| Biometric | | | | not believe someone's |
| information- | | Behavior of | Doubt, | speaking, feel |
| eyes | Eyes_frowning | squinting | Discomfort | uncomfortable |
| Biometric | Eyes_wide | Behavior of | Surprise, | When telling |
| information- | open | opening eyes | Fear | shocking story, when |
| eyes | | wide | | hearing interesting |
| | | | | story |
| Biometric | Pupil dilation | Degree of | Interest, | Pupils dilate in |
| information- | | pupil dilation | Tension | emotionally agitated |
| eyes | | | | state |
| Biometric | Cheek_puffed | Behavior of | Confidence, | Express |
| information- | | puffing cheeks | Exaggerated | confidence by puffing |
| face | | | expression | cheeks |
| Biometric | Cheek_frowning | Motion of | Doubt, | Frown cheeks in |
| information- | | frowning cheeks | Discomfort | uncomfortable |
| face | | | | conversation |
| Biometric | Chin_direction | Measure | Determination, | Fix chin while |
| information- | | movement of | Tension | making decision |
| face | | chin | | |
| Biometric | Chin_direction | Behavior of | Concentration, | Push chin forward |
| information- | | pushing chin | Will | expressing strong will |
| face | | forward | | |
| Biometric | Jaw_opened | Behavior of | Surprise, | State of being |
| information- | | opening mouth | Intention of | surprised or before |
| face | | | speaking | speaking |
| Biometric | Mouth_direction | Maintain lips | Neutral state | State of not |
| information- | | basically | | speaking to keep |
| face | | | | neutral position |
| Biometric | Mouth_closed | Behavior of | Silence, | Close lips and |
| information- | | closing mouth | Resistance, | keep silent |
| face | | | Oppression | |
| Biometric | Mouth_dimple | Dimples | Satisfaction, | Dimples are |
| information- | | shown on both | Pride | shown in satisfied |
| face | | sides of lips | | state |
| Biometric | Mouth_corners | Behavior of | Sadness, | Mouth corners |
| information- | | turning mouth | Disappointment | turn down in sadness |
| face | | corners down | | |
| Biometric | Mouth_narrowed | Motion of | Doubt, | Narrow lips with |
| information- | | narrowing mouth | Wariness | caution |
| face | | | | |
| Biometric | Mouth_lowered | Motion of | Disappointment, | Lower lip turns |
| information- | | turning lower lip | Resignation | down in |
| face | | down | | disappointment |

TABLE 3

| | | | | Maintain oxygen |
|---|---|---|---|---|
| Biometric | Oxygen | Ratio of oxygen | Healthy | saturation in healthy |
| information | saturation | saturation in blood | state | state |
| Biometric | Change in | Change in facial | Expression | Express emotion |
| information | facial | expression due to | of emotion | by laughter |
| | expression | change in facial | | |
| | | muscles | | |
| Behavioral | Change in | Change in posture | Discomfort, | Change posture in |
| information | body posture | of whole body | Tension | tensed state |
| Behavioral | Body | Frequency and | Active | Keep moving body |
| information | movement | pattern of body | | in anxiety |
| | | movement | | |
| Voice | Pitch of | Changes in tone | Emphasis, | Pitch of voice goes |
| information | voice | and pitch of voice | Expression of | up when exited |
| | | | emotion | |
| Voice | Speed of | Pace and speed of | Tension, | Speak faster when |
| information | speech | speech | Expression of | nervous |
| | | | emotion | |
| Voice | Intonation | Pattern of | Emphasis, | Intonation changes |
| information | of speech | intonation of speech | Emotional | when emphasizing |
| | | | state | |
| Voice | Tremor of | Tremors revealed | Tension, | Voice trembles |
| information | voice | in voice | Anxiety | with tension |
| Voice | Voice | Information | Information | Transfer positive |
| information | information | included in voice | transfer | information on |
| | | | | specific subject |
| Language | Analysis | Subject and | Interest in | Express interest in |
| information | of speech | content revealed in | subject | subject |
| | | speech | | |
| Language | Pattern of | Pattern of words | Thinking | Show interest |
| information | words used | repeatedly used in | pattern | through repeated |
| | | speech | | words |
| Language | Complexity | Structural | Cognitive | Reveal thinking |
| information | of sentence | complexity of | ability | using long sentence |
| | | sentence | | |
| Language | Frequency | Frequency of | Usage | Frequently use |
| information | of words | using specific words | habit | specific words |
| Language | Preferred | Most preferred | Preference, | Repeat easy |
| information | subject | subject | Avoidance | subjects to avoid |
| | | | | frequently discussed |
| | | | | subject and |
| | | | | uncomfortable |
| | | | | conversation |
| Language | Rejected | Subject showing | Rejection, | Subject desired to |
| information | subject | rejection | Discomfort, | avoid |
| | | | Defensiveness | |
| Language | Key | Important subject | Key point | Key point of |
| information | subject | in conversation | of discussion | conversation |

Meanwhile, the indicator data shown in Tables 1 to 3 are only examples, and the indicator data, i.e., the responses of the examinee quantitatively converted by the apparatus 100, is not limited to the embodiment described above.

At step S1030, the apparatus 100 may determine the statistical distribution position of the response of the examinee by comparing the indicator data of the examinee with previously constructed normative data where a plurality of responses to the Rorschach test is configured as statistical data.

In order to construct the normative data, the apparatus 100 may collect response data including verbal responses, behavioral responses, and biometric indicators collected from a preset number of experimenters or more for each card included in the Rorschach test before the operation of FIG. 2 is performed or at step S1030 itself, and construct the normative data by converting the probability of specific response data to be appeared on each card of the Rorschach test into a percentile or a standard score on the basis of the response data.

For example, in order to construct the normative data, the apparatus 100 may collect results of performing Rorschach tests on a predetermined number of normal groups and on groups having specific psychological characteristics, and statistically analyze the response pattern that appears on each card. In this process, the apparatus 100 may statistically analyze the frequency of appearance of a response type for each card according to various examinees. Based on the data collected in this way, the apparatus 100 may normalize distribution of the responses to each card, compute the probability of a specific response to be appeared in the normal groups and the groups having specific psychological characteristics, and convert the probability into a percentile score or a standard score. Through this, the apparatus 100 may construct normative data for each card, and evaluate the response of the examinee who perform the Rorschach test by comparing the response with corresponding normative data.

As the normative data is constructed, at step S1030, the apparatus 100 may compare the similarity between the response of the examinee and those of the specific group using data on the normal groups and the groups having specific psychological characteristics included in the normative data, and compare the indicator data of the examinee by converting it into a normal distribution or a percentile score of the normative data. For example, when the examinee shows a verbal response of “It's like a dark picture” to a particular card, has a gaze time longer than the average, and shows a behavioral response of dilated pupils and a biometric indicator of increasing skin conductance response, the apparatus 100 may evaluate to which of the normal groups and the groups having specific psychological characteristics these responses are more similar. To this end, the apparatus 100 may compute the probability of the corresponding response to be appeared in a specific group by analyzing the response patterns of existing examinees stored in the normative data, and compare response data of the examinee with the probability value.

For example, the apparatus 100 may generate statistical distribution information on how much the response of the examinee is different from the average response pattern of a normal group on the basis of the comparison result using the normative data. For example, when the response of the examinee is compared with the average response pattern of a normal group and is equal to or higher than 85% of the percentile score, the apparatus 100 may output that the response is an unusual response outside the normal range. In addition, when the probability of the response of the examinee to be appeared in a group having specific psychological characteristics on the basis of the normative data, the similarity with the group may be output after being converted into a percentile score or a standard score.

Based on the result, the apparatus 100 may evaluate whether the response of the examinee is similar to a general card response pattern or reflects a specific psychological characteristic through the neural network model described below, and more objective psychological interpretation can be made on the basis of the evaluation.

At step S1040, the apparatus 100 may generate a projection index that indicates whether the response of the examinee is due to the physical characteristics of the card or the psychological projection as a numerical value from the indicator data and the statistical distribution position using a previously learned neural network model.

The projection index is an indicator that analyzes the response of an examinee and probabilistically evaluates whether the response is derived by the physical characteristics of the Rorschach card itself (e.g., contrast, symmetry, shape, etc.) or generated by the internal psychological state of the examinee and a projective mechanism.

To this end, the apparatus 100 may input the indicator data generated at step S1020 and the statistical distribution position computed at step S1030 into the neural network model, and output a projection index including a probability value of whether the response of the examinee is due to the physical characteristics of the card or the psychological projection.

For example, when an examinee shows a response of “like a butterfly” to a particular card, the neural network model may compare the frequency of a response such as “butterfly” to the card in the normative data with the response data of the examinee. Through this, a probability value indicating whether the response is derived by the morphological characteristics of the card (e.g., curve, dot arrangement, contrast, etc.) or by the individual psychological factors of the examinee (e.g., anxiety, obsession, projection of a specific experience, etc.) may be computed.

The apparatus 100 may provide the computed projection score after converting it into a value greater than or equal to 0 and smaller than or equal to 100, or a percentile score. For example, when the probability of a specific response for being derived by the physical characteristics of the card is 70% and the probability to be appeared by psychological projection is 30%, the apparatus 100 may set the projection index of the response to 30.

For example, the design and learning of the neural network model may be performed as described below.

A neural network model is designed to analyze response data of an examinee and compute a projection index. For example, as the neural network model, a model based on a multilayer perceptron (MLP), convolutional neural network (CNN), or Transformer-based model may be applied. Selection of the model may be optimized according to the characteristics of verbal responses, behavioral responses, and biometric indicators, and in order to effectively process multimodal data, the model may be designed in a way of applying an individual network structure to be suitable for each data type, and then finally performing integrated analysis. Accordingly, the neural network model may be configured of an input layer, several hidden layers, and an output layer.

As the indicator data (verbal responses, behavioral responses, and biometric indicators) of the examinee generated at step S1020 and the statistical distribution position computed at step S1030 are input into the input layer of the neural network model, the neural network model may be trained. For example, verbal response data may be input in the form of a vector reflecting the results of morphological analysis and sentiment analysis, and behavioral response data may be input as a value of gaze pattern, response time, card rotation, or the like, and in addition, the biometric indicator may be input after being quantified as an indicator value of heart rate, skin conductance response, change in the pupil size, or the like.

In the output layer of the neural network model, learning data where the projection index of each experimenter is labeled with a value greater than or equal to 0 and smaller than or equal to 100 or a percentile score may be used for learning so that the projection index of the experimenters for the normal group and the group having specific psychological characteristics may be used as a correct answer value of each card.

Learning of the neural network model may be performed in a supervised learning mode. To this end, cross entropy or mean squared error may be used as a loss function, and stochastic gradient descent or Adam may be applied as an optimization algorithm. Accordingly, on the basis of a response of an examinee input at the inference step, the learned model computes a probability value of whether the response is derived by the physical characteristics of the card or generated by psychological projection, and based on this, the model may output a final projection index.

Meanwhile, in outputting the projection index, the apparatus 100 may analyze the statistical position of the projection index of the examinee with reference to the percentile information or standard score of the statistical projection index of a specific Rorschach card from the normative data, and compute a final projection index by comprehensively evaluating the test result of each card and all test results on the basis of the analyzed data.

For example, when an examinee shows a response of “like a butterfly” to a particular card, the apparatus 100 may analyze how frequently previous examinees have shown similar responses to the card, and convert the frequency of the response shown in the entire dataset into a percentile or a standard score. When the response appears at a frequency of less than 10% in the general normal group, but appears at a frequency of 70% or more in a group having specific psychological characteristics, the apparatus 100 may evaluate that the response is highly probable to be related to a specific psychological tendency. Through this, the apparatus 100 may determine the statistical distribution position of the response and quantitatively evaluate how much the response is different from the average response pattern of the normal group by comparing the response of the examinee with existing data.

In addition, the apparatus 100 may provide the computed projection index as a numerical result so that the examiner may interpret. In this way, as the responses of the examinee are quantitatively quantified, the apparatus 100 may make an objective assessment on whether the responses shown in the Rorschach test are similar to responses to general cards or individual psychological factors are applied to the responses. Through this, the examiner may numerically grasp psychological projection strength of the examinee, and as a result, consistency and reliability of interpretation can be enhanced.

According to the embodiment described above, the present invention may make a more objective and quantitative psychological assessment by automating the implementation, scoring, and interpretation of the Rorschach test to utilize the Rorschach test based on projection index. While conventional test methods greatly rely on subjective interpretation of an examiner, the present invention may quantitatively compute projection indexes by comprehensively analyzing biometric indicators and verbal and behavioral responses of an examinee.

In addition, as the projection indexes computed through the present invention can be used for more precise analysis of psychological characteristics compared with conventional subjective assessment methods, reliability and consistency of the test results can be improved, and based on this, more scientific diagnosis and intervention can be made without intervention of an expert in the field of mental health management and psychotherapy.

Through this, the present invention promotes popularization of the psychological assessment and allows many people to receive more reliable psychological analysis.

It should be understood that various embodiments of this document and the terms used herein are not intended to limit the technical features described in this document to specific embodiments, but include various modifications, equivalents, or substitutes of the embodiments. In connection with the description of drawings, similar reference numerals may be used for similar or related components. The singular form of a noun corresponding to an item may include one or more items, unless the related context clearly indicates otherwise.

In this document, each of phrases such as “A or B”, “at least one among A and B”, “at least either A or B”, “A, B, or C”, “at least one among A, B, and C”, and “at least either A, B, or C” may include all possible combinations of the items listed together in a corresponding phrase among the phrases. Terms such as “1st”, “2nd”, “first”, or “second” may be used only to distinguish a corresponding component from another corresponding component, and do not limit the components in any other aspect (e.g., importance or order). When a certain (e.g., a first) component is referred to as being “coupled” or “connected” to another (e.g., a second) component with or without a term such as “functionally” or “communicatively”, it means that the component may be connected to another component directly (e.g., wired), wirelessly, or through a third component.

The term “module” used in this document may include a unit implemented in hardware, software, or firmware, and may be used interchangeably with terms such as logic, logic block, part, or circuit. A module may be an integrally configured component, or a minimum unit of a component or a portion thereof that performs one or more functions. For example, according to an embodiment, a module may be implemented in the form of an application-specific integrated circuit (ASIC).

Various embodiments of this document may be implemented as software (e.g., a program) including one or more commands stored in a storage medium (e.g., a memory) that can be read by a device (e.g., an electronic device). The storage medium may include a random-access memory (RAM), a memory buffer, a hard drive, a database, an erasable programmable read-only memory (EPROM), an electrically erasable read-only memory (EEPROM), a read-only memory (ROM), and/or the like.

In addition, the processor in the embodiments of this document may call at least one command among one or more stored commands from the storage medium and execute the command. This allows the device to operate to perform at least one function according to the called at least one command. The one or more commands may include a code generated by a compiler or a code that can be executed by an interpreter. The processor may be a general-purpose processor, a Field Programmable Gate Array (FPGA), an Application Specific Integrated Circuit (ASIC), a Digital Signal Processor (DSP), and/or the like.

The storage medium that can be read by a device may be provided in the form of a non-transitory storage medium. Here, ‘non-transitory’ only means that the storage medium is a tangible device and does not include signals (e.g., electromagnetic waves), and this term does not distinguish the cases where data is stored semi-permanently on the storage medium from the cases where data is stored temporarily.

The method according to various embodiments disclosed in this document may be provided to be included in a computer program product. The computer program product may be traded between a seller and a buyer as goods. The computer program product may be distributed in the form of a machine-readable storage medium (e.g., a compact disc read only memory (CD-ROM)), or may be distributed online (e.g., downloaded or uploaded) through an application store (e.g., Play Store) or directly distributed between two user devices (e.g., smartphones). In the case of online distribution, at least a part of the computer program product may be at least temporarily stored in a machine-readable storage medium, such as a memory of a manufacturer's server, an application store's server, or a server, or may be temporarily generated.

According to various embodiments, each component (e.g., a module or a program) of the components described above may include a single or a plurality of entities. According to various embodiments, one or more of the components or operations of the components described above may be omitted, or one or more other components or operations may be added. Alternatively or additionally, a plurality of components (e.g., modules or a programs) may be integrated into a single component. In this case, the integrated component may perform one or more functions of each of the plurality of components in a way identical or similar to those performed by the corresponding component among the plurality of components before the integration. According to various embodiments, the operations performed by the modules, programs, or other components may be executed sequentially, in parallel, repeatedly, or heuristically, or one or more of the operations may be executed in a different order or omitted, or one or more other operations may be added.

### DESCRIPTION OF SYMBOLS

100: Apparatus
110: Memory
120: Processor
130: Input/Output Interface
140: Communication Interface

What is claimed is:

1. A method performed by a Rorschach test projection index computing apparatus operated by a processor, the method comprising:
an operation of acquiring a response of an examinee including a verbal response, a behavioral response, and a biometric indicator, while the examinee performs a Rorschach test;
an operation of generating indicator data obtained by quantitatively converting each of the verbal response, behavioral response, and biometric indicator of the examinee;
an operation of determining a statistical distribution position of the response of the examinee by comparing the indicator data with previously constructed normative data where a plurality of responses to the Rorschach test is configured as statistical data; and
an operation of generating a projection index that indicates whether the response of the examinee is due to physical characteristics of a card or psychological projection as a numerical value from the indicator data and the statistical distribution position using a previously learned neural network model.

2. The method according to claim 1, wherein the operation of acquiring a response of an examinee includes an operation of presenting a Rorschach card to the examinee and acquiring an answer to what the presented card looks like, and additionally presenting preset questions about the presented card and acquiring responses to the questions.

3. The method according to claim 2, wherein the operation of acquiring a response of an examinee includes:
an operation of converting voices of the examinee into text data through voice recognition and natural language processing as the verbal response;
an operation of measuring a gaze pattern of the examinee, whether the card is rotated, and a response time using a webcam to acquire the behavioral response; and
an operation of measuring biometric indicators including electromyography, skin conductance response, peripheral body temperature, heart rate, respiration rate, electroencephalography, and eye movement of the examinee using biometric sensors to acquire the biometric indicators.

4. The method according to claim 3, wherein the operation of generating indicator data includes:
an operation of generating the indicator data by tokenizing the text data into units of morphemes using a morphological analyzer, tagging each token with a part of speech, and removing particles and conjunctions;
an operation of detecting behavioral patterns including eyebrow movement, lip movement, eye blink, eye movement, pupil dilation, facial movement, neck movement, upper body movement, and lower body movement set in advance among the behavioral response using an image analysis technique; and
an operation of generating indicator data by converting the biometric indicators into quantitative values using a signal processing technique.

5. The method according to claim 1, wherein the operation of evaluating a statistical distribution position includes:
an operation of collecting response data including a verbal response, a behavioral response, and a biometric indicator from a preset number of experimenters or more for each card included in the Rorschach test; and
an operation of previously constructing normative data by converting a probability of specific response data to be appeared on each Rorschach card into a percentile or a standard score on the basis of the collected response data.

6. The method according to claim 5, wherein the operation of evaluating a statistical distribution position includes:
an operation of comparing a similarity between the response of the examinee and those of a specific group using data on a normal group and a group having specific psychological characteristics included in the normative data;
an operation of comparing the indicator data of the examinee by converting it into a normal distribution or a percentile score of the normative data; and
an operation of outputting how much the response of the examinee is different from an average response pattern of the normal group on the basis of a result of the comparison.

7. The method according to claim 1, wherein the operation of generating a projection index includes:
an operation of receiving the indicator data and the statistical distribution position and comparing them with the normative data, by the neural network model;
an operation of computing a probability value of whether the response of the examinee is due to the physical characteristics of the card or the psychological projection on the basis of the normative data; and
an operation of computing a projection index of the examinee by converting the probability value into a percentile or a standard score.

8. The method according to claim 7, wherein the operation of computing a probability value includes:
an operation of converting the indicator data of the examinee into a feature vector; an operation of computing a probability of a response that may appear on a specific card by comparing the feature vector with existing indicator data of the examinee included in the normative data; and
an operation of evaluating whether the response of the examinee is a general card characteristic response or a psychological projective response on the basis of a result of the comparison.

9. The method according to claim 8, wherein the operation of computing a projection index includes:
an operation of analyzing a statistical position of the projection index of the examinee with reference to the percentile information or standard score of the statistical projection index of a specific Rorschach card from the normative data;
an operation of computing a final projection index by comprehensively evaluating a test result of each card and all test results on the basis of the analyzed data; and
an operation of providing the computed projection index as a numerical result so that the examiner may interpret.

10. A Rorschach test projection index computing apparatus comprising:
a memory including instructions; and
a processor that performs a predetermined operation based on the instructions, wherein the operation of the processor includes:
an operation of acquiring a response of an examinee including a verbal response, a behavioral response, and a biometric indicator, while the examinee performs a Rorschach test;
an operation of generating indicator data obtained by quantitatively converting each of the verbal response, behavioral response, and biometric indicator of the examinee;
an operation of determining a statistical distribution position of the response of the examinee by comparing the indicator data with previously constructed normative data where a plurality of responses to the Rorschach test is configured as statistical data; and
an operation of generating a projection index that indicates whether the response of the examinee is due to physical characteristics of a card or psychological projection as a numerical value from the indicator data and the statistical distribution position using a previously learned neural network model.
